(19) United States
(12) Patent Application Publication
(10) Pub. No.: US 2006/0167281 A1
(43) Pub. Date: Jul. 27, 2006

(54) 1,2,4- Trioxepanes as precursors for lactones

(76) Inventors: John Meijer, Deventer (NL); Rolf Hendrik Van Den Berg, Kring van Dorth (NL)

Correspondence Address: OLIFF & BERRIDGE, PLC, P.O. BOX 19928, ALEXANDRIA, VA, 22320, US

(21) Appl. No.: 10/564,554

(22) PCT Filed: Jul. 12, 2004
(86) PCT No.: PCT/EP04/07839

§ 371 Date: Feb. 2, 2006

(30) Foreign Application Priority Data
Jul. 17, 2003 (EP) 03077238.8
Sep. 2, 2003 (US) 60499415

(51) Int. Cl. C07D 307/02

(52) U.S. Cl. 549/295

(57) ABSTRACT

The present invention pertains to a novel process for the preparation of lactones by decomposition of a 1,2,4-trioxepane of formula (I) wherein, R is H or CH₃, n is 1-14, Rx independently is any substituent on the ring structure, including substituents which form bi- or tricyclic structures, and m is 0-34.

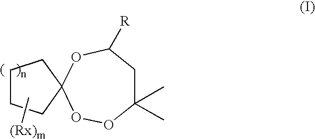

The present invention relates to a process for the preparation of lactones and the use of these compounds as perfuming agent or odorant.

Well-known methods for the production of macrocyclic lactones is the thermal decomposition and the photolysation of di- and trimeric cyclic peroxides. For example, U.S. Pat. No. 3,528,898 describes both the thermal decomposition of di- or triperoxides by heating to a temperature above 100° C. and the photochemical decomposition of such peroxides by irradiation of the diperoxide in a suitable solvent with ultra-violet light from a mercury lamp or other convenient source. Thermal decomposition of peroxides is disadvantageous, however, in that the thermal reaction is difficult to control and susceptible to explosions. In order to avoid explosions as much as possible, the decomposition must be carried out in the presence of high amounts of solvents such as methanol and benzene. The photolytic process must also be carried out cautiously at high dilutions. Hence, large quantities of diluent are required. Another disadvantage is that often expensive and bulky equipment must be employed. Furthermore, in the thermolytic process mixtures of macrocyclic hydrocarbons and lactones are obtained wherein the proportions of lactones are relatively small. In the photolytic process also mixtures of macrocyclic lactones and hydrocarbons are obtained, both in relatively low yields.

U.S. Pat. No. 3,960,897 relates to the thermolytic decomposition of dicycloalkylidene and tricycloalkylidene cyclic peroxide compounds into macrocyclic hydrocarbons and lactones. In said document, it is described that the inclusion of relatively large amounts of alkane solvent in the thermolytic decomposition media helps to avoid explosions. Moreover, the addition of these alkane solvents appeared to lead to increased yields of the macrocyclic compounds and an increase in the proportion of the lactone component in the mixture. However, the yields of lactone generally still do not exceed 20-25% of the theoretical yield, whereas the yields of the macrocyclic hydrocarbons in general also are not higher than 20-25%. In the process according to U.S. Pat. No. 3,960,897, a mixture of peroxide and alkane solvent is heated to a temperature of about 100-350° C., preferably about 180° C., at which the decomposition takes place. The reaction times vary from a few minutes to several days. Alkane solvents which were employed include linear alkanes such as decane, nonane, dodecane, undecane, etc., or the branched alkanes Isopar® H or K. The amount of alkane solvent employed is preferably about 4 to 8 parts by weight of solvent per part of peroxide starting material. The macrocyclic lactones produced in the above-described process can be used as perfuming agents. The macrocyclic hydrocarbons produced in admixture with said lactones are only suitable for use in the perfume industry after they have been oxidised.

It is an object of the present invention to provide an improved process for the preparation of lactones which gives good yields of the desired compounds. Surprisingly, we have found that by thermal decomposition of 1,2,4-trioxepanes, the corresponding lactones can be obtained in good to excellent yields. Moreover, said process is convenient, safe, and commercially attractive because of the readily accessible starting materials and the good yields of the desired lactones.

Accordingly, the present invention relates to a process for the thermolytic decomposition of 1,2,4-trioxepanes into lactones. In addition, the present invention relates to the use of these lactones as perfuming agent or odorant.

The 1,2,4-trioxepanes employed as starting materials in the process of the present invention are represented by formula (I):

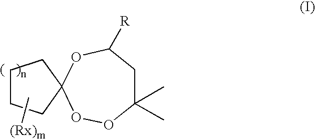

wherein
R is H or CH₃;
n is 1-14;
Rx independently is any substituent on the ring structure, including substituents which form bi- or tricyclic structures; and
m is 0-34.

Preferably, each Rx is independently selected from the group consisting of a hydrogen, hydroxy, halogen, alkoxy, acyloxy, carboxyl, hydroxyalkyl, haloalkyl, alkoxy alkyl, acyloxy alkyl, acyloxy aryl, carboxyl aryl, amido, amino, amino alkyl, and amino aryl group. The alkyl groups and substituted alkyl groups are linear or branched and preferably are C₁-C₈ alkyl groups, more preferably C₁-C₅ alkyl groups. Said aryl groups preferably are monocyclic aryl groups. n preferably is 1-8 and most preferably 2-8. m preferably is 0-22, more preferably 1-20, and most preferably 2-16.

1,2,4-Trioxepanes according to formula (I) have a good shelf-life stability and are relatively safe to handle. Furthermore, they are easily accessible. They can be prepared by various methods known in the literature. For example, in Physical Organic Chemistry, 1986, Vol. 31, pp. 113-120, M. Kobayashi et al. describe several routes towards 1,2,4-trioxepanes. The most preferred method for the preparation of the 1,2,4-trioxepanes according to the present invention, however, is the reaction between a cyclic ketone and a hydroperoxide compound. The latter preparation method can be found in WO 98/50354, which relates to a process for cross-linking thermoplastic polymers. In this document, the preparation of a 1,2,4-trioxepane from hexyleneglycol hydroperoxide and cyclohexanone is described.

Particularly preferred 1,2,4-trioxepanes according to the invention are, but are not limited to, the reaction products of hexyleneglycol hydroperoxide or isopreneglycol hydroperoxide (HOOC(CH₃)₂CH₂CH₂OH) with a compound selected from the group consisting of cyclobutanone, cyclopentanone, cyclohexanone, cycloheptanone, cyclooctanone, cyclononanone, cyclodecanone, cycloundecanone, cyclododecanone, cyclotridecanone, cyclotetradecanone, cyclopentadecanone, cyclohexadecanone, cycloheptadecanone, cyclooctadecanone, camphor, norbornanone, ethyl2-oxocyclopentylacetate, ethyl6-(2-oxocyclopentyl)hexanoate, 3-methylcyclopentanone, fenchone, 2-methylcyclopentanone, methyl2-cyclopentanonecarboxylate, 4-t-butylcyclohexanone, menthone, 2-methylcyclohexanone, 3-methylcyclohexanone, 2-phenylcyclohexanone, 3,3,5,5-tetramethylcyclohexanone, 2,6-dimethylcyclohexanone, bicylo[3.2.1]octan-2-one, 2B-cyanoethylcyclohexanone, 4-ethylcyclohexanone, bicyclo[3.3.1]nonan-9-one, dihydrocarvone, 2-t-butylcyclohexanone, 3,3,5-trimethylcyclohexanone, 6-carbethoxy-2,6,6-trimethylcyclohexanone, 2,6,6-trimethylcyclohexanone, 2-ethoxycyclohexanone, 2,2,6,6-tetramethylcyclohexanone, 3-methylene-2-norbornanone, pulegone, and ethyl 2-oxo-1-cyclooctanecarboxylate. Especially preferred 1,2,4-trioxepanes are the reaction product of hexyleneglycol hydroperoxide and cyclohexanone and the reaction product of hexyleneglycol hydroperoxide and cycloheptanone.

The lactones according to the present invention can be obtained by decomposition of the above-described 1,2,4-trioxepanes. Any conventional procedure for achieving decomposition of organic compounds known to the person skilled in the art can be used, as long as that procedure results in the formation of the lactones according to the present invention. Preferably, the lactones are obtained via thermal decomposition of the above-described 1,2,4-trioxepanes. In a particularly preferred embodiment, the decomposition process comprises the steps of
(a) heating a small amount of a suitable medium to a temperature at which the 1,2,4-trioxepane of formula (I) which is to be the subject of the decomposition reaction decomposes, and
(b) subsequently adding said 1,2,4-trioxepane to the preheated medium.

The trioxepane cannot be added at once because of the exothermic nature of the decomposition reaction. Hence, the supply of the starting material occurs at a rate which enables the skilled person to control the temperature and maintain the whole at the temperature referred to above.

If the 1,2,4-trioxepane compound is a liquid at room temperature, it is preferably added to the previously heated medium in the undiluted, neat form. However, it is also possible to mix the trioxepane compound with a minimum amount of a suitable solvent and slowly add the resulting mixture to the previously heated small amount of medium. If the 1,2,4-trioxepane compound is a solid at room temperature, it can be added in the molten state or dissolved in a minimum amount of a suitable solvent to the previously heated amount of medium. It is noted that 1,2,4-trioxepanes, like most organic peroxides, are potentially shock-, heat-, and friction-sensitive and therefore should be handled with care.

Preferably, the medium is a solvent. Solvents which are suitable for use in the decomposition process according to the invention comprise linear or branched alkane solvents, such as nonane, decane, undecane, dodecane, paraffin oil, Isopar® solvents, Shellsol® solvents or a mixture thereof. Particularly preferred solvents are the Isopar® solvents, especially Isopar® H. Other solvents which are suitable for use in the decomposition process according to the invention are aromatics such as toluene, xylene, cumene, ethylbenzene, cumene, p-cumene, pseudocumene, mesitylene, o-, or p-diisopropylbenzene, tetrahydro-naphthalene, chlorobenzene, o-dichlorobenzene, anisole; alcohols such as amylalcohol, hexanol, heptanol, octanol, 2-ethylhexanol, 3,5,5-trimethylhexanol, isooctanol, cyclohexanol, benzylalcohol, ethyleneglycol, ethylcellosolve, butylcellosolve, propyleneglycol methyl ether, esters such as butylacetate, 2-ethylhexylacetate, butylcellosolve acetate, benzylacetate, methylethyl acetoacetate, ethyl acetoacetate; ethers such as diglyme, triglyme; amines such as N,N-diethyleaniline, benzylamine, N-methylbenzylamine, N,N-dimethylbenzylamine.

The amount of medium to which the 1,2,4-trioxepane is added for the thermolytic decomposition reaction according to the present invention preferably is small. By a small amount of medium is meant an amount which is at least about 0.01 part by weight of medium per part by weight of 1,2,4-trioxepane starting material, more preferably at least about 0.05 part by weight, and most preferably at least 0.1 part by weight, whereas the preferred maximum amount of medium does not exceed 1.5 parts by weight of medium per part by weight of 1,2,4-trioxepane starting material, more preferably 1.0 part by weight, and most preferably 0.5 part by weight of medium per part by weight of 1,2,4-trioxepane. It is also possible to apply amounts of medium exceeding 1.5 parts by weight of medium per part by weight of 1,2,4-trioxepane starting material in the process according to the present invention, but such large quantities of medium are less preferred, because the process will be economically less attractive.

The process of the present invention occurs at a temperature at which the 1,2,4-trioxepane which is subject to decomposition readily decomposes. Obviously, this temperature varies with the particular 1,2,4-trioxepane used in the process. However, in general, the process occurs at a temperature in the range of between 100 and 400° C. More preferably, the process according to the invention is performed at a temperature between 100 and 300° C. Most preferred is a reaction temperature between 120 and 250° C.

The 1,2,4-trioxepanes according to the invention decompose when added to a small amount of a suitable medium at raised temperature to yield a mixture of compounds of the general formulae as depicted in Scheme 1. 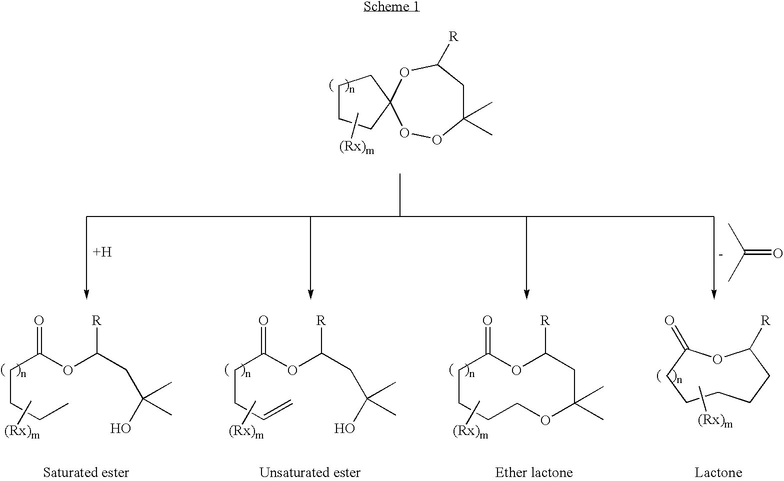
wherein R, n, Rx, and m have the values noted above.

The major component in the reaction mixture is the corresponding ether lactone. The amount of ether lactone present in the reaction mixture varies. Said compound is normally present in an amount of at least 20 wt %, preferably at least 30 wt %, and most preferably in an amount of at least 40 wt %, based on the total weight of the monomeric products.

The corresponding lactone is formed under the elimination of acetone. The lactone is also present in the product mixture in a relatively large amount. Normally, said compound is present in the mixture in an amount of at least 15 wt %, more preferably, 25 wt %, and most preferably at least 35 wt %, based on the total weight of the monomeric products.

Other products which are formed by thermal decomposition of the 1,2,4-trioxepanes according to the present invention are a saturated ester compound and an unsaturated ester compound. These two compounds are only present in a minor amount. Normally, the amount of these two products does not exceed 20 wt %, preferably 10 wt %, more preferably 6 wt %, and most preferably 3 wt, based on the total weight of the monomeric products.

In addition to the monomeric products just-described, oligomeric products might be formed during the thermal decomposition process. Normally, the amount of oligomeric products does not exceed 30 wt %, preferably 20 wt %, more preferably 10 wt %, and most preferably 5 wt %, based on the total weight of all reaction products.

When the decomposition is complete, any solvent which may be present in the reaction mixture is evaporated. Subsequently, the reaction mixture may be distilled and the crude (macrocyclic) lactone and the crude ether lactone are isolated. If necessary, the crude product can be further purified, e.g. by crystallization.

Macrocyclic lactones such as d,l-muscone(3-methylcyclopentadecanone), cyclopentadecanone, cyclopentadecanolide, and cyclohexadecanolide have distinct and pronounced musk-like odours. They are therefore frequently employed as synthetic musks in the perfume or odorant industry. Macrocyclic ether lactones and macrocyclic anhydrides are also known to have characteristic musk-like odours and hence they are employed as synthetic musks as well. The (macrocyclic) lactones and ether lactones obtained by the process according to the present invention are therefore suitable for use in fragrance applications.

The present invention is elucidated by means of the following non-limiting Examples.

### EXAMPLE 1

In this Example, the preparation of C12 ether lactone is described (see also Scheme 2):

A 1 litre reactor was charged with 100 g Shellsol® D60 and heated to 195° C. Cyclohexanone trioxepane (235 g) was dosed in 90 minutes while stirring and the temperature was kept at 190-195° C. under distilling off of the volatile components. The obtained reaction mixture was stirred for an additional 15 minutes at 190° C.

The remaining reaction mixture was fractionated at 2 mm Hg pressure. The main fraction (weight 110.7 g), distilled at 97-98° C., contained 90.0% area of C12 etherlactone as analysed by GC.

100 g of the main fraction were recrystallised from 200 g ethanol, after filtration over a G-3 glass filter, and washed once with ethanol. The filter cake was dried in the air at room temperature during 24 hrs, weight: 75.0 g, analysed by GC: 99.9% area.

The melting point was determined by DSC at 5° C./minute: 57.4° C.

The C12 etherlactone was characterised by GC-MS and NMR.

In the crude reaction mixture before distillation the following compounds were identified by GC-MS:

saturated C12 ester, C12 unsaturated ester, C9 lactone, and C12 ether lactone.

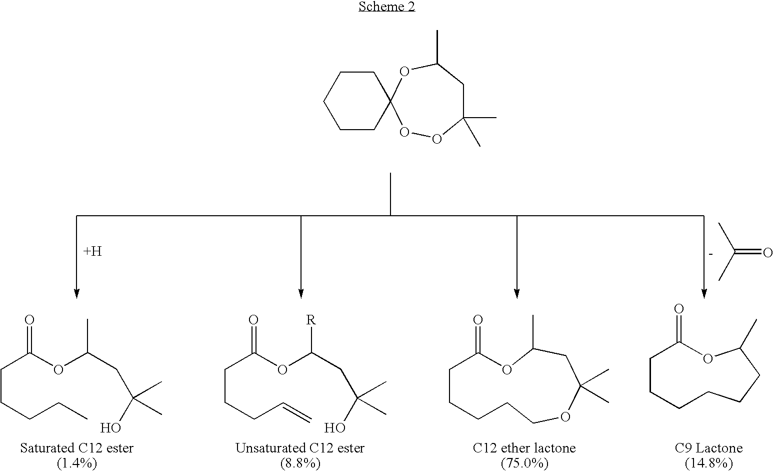

### EXAMPLE 2

In this Example, the preparation of C11 ether lactone is described:

A 100 ml 3-necked flask, provided with a stirrer, a dosing funnel, a thermometer, and a distillation set-up was charged with 5 g Isopar H. After heating to 160° C., 40 g cyclopentanone trioxepane were dosed, while the temperature was kept below 190° C., and 11.0 g of distillate were obtained (during decomposition).

The crude reaction mixture before distillation (31.3 g) was analysed by GC using 2 internal standards. It was found that 27.9% w/w (response factor 1.5) of the C11 lactone was present in the reaction mixture, as characterised by GC-MS.

### EXAMPLE 3

C13 ether lactone was prepared analogously to the preparation of C11 ether lactone as described in Example 2, using 20 g Isopar H and 71.8 g cycloheptanone trioxepane.

The crude reaction mixture before distillation (65.7 g) was analysed by GC using 2 internal standards. It was found that 43.2% w/w (response factor 1.5) of the C13 ether lactone was present in the reaction mixture, as characterised by GC-MS.

### EXAMPLE 4

C14 ether lactone was prepared analogously to the preparation of C11 ether lactone as described in Example 2, using 10 g Isopar H and 40.0 g cyclooctanone trioxepane.

The crude reaction mixture before distillation (33.2 g) was analysed by GC using 2 internal standards. It was found that 36.6% w/w (response factor 1.5) of the C14 ether lactone was present in the reaction mixture, as characterised by GC-MS.

### EXAMPLE 5

C18 ether lactone was prepared analogously to the preparation of C11 ether lactone as described in Example 2, using 5 g Isopar H and 30.0 g cyclododecanone trioxepane in 15 g Isopar H (preheated to 50° C.).

The crude reaction mixture before distillation (23.5 g) was analysed by GC using 2 internal standards. It was found that 47.3% w/w (response factor 1.5) of the C18 ether lactone was present in the reaction mixture, as characterised by GC-MS.

1. A process for the preparation of lactones by decomposition of a 1,2,4-trioxepane according to formula (I) 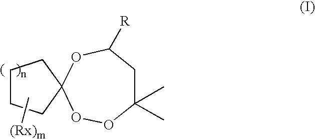
wherein
R is H or CH₃;
n is 1-14;
Rx independently is any substituent on the ring structure, including substituents which form bi- or tricyclic structures; and
m is 0-34.

2. A process for the preparation of lactones according to claim 1 comprising the steps of
(a) heating a small amount of a suitable medium to the temperature at which the 1,2,4-trioxepane decomposes, and
(b) subsequently adding said 1,2,4-trioxepane to the preheated amount of medium while controlling the reaction temperature.

3. A process for the preparation of lactones according to claim 2 wherein the medium is a linear or branched alkane solvent, preferably selected from the group consisting of nonane, decane, undecane, dodecane, paraffin oil, Isopar® solvents, and Shellsol® solvents.

4. A process for the preparation of lactones according to claim 3 wherein the solvent comprises an Isopar® solvent, preferably Isopar® H.

5. A process for the preparation of lactones according to claim 1 wherein the small amount of medium is between 0.01 and 1.5 parts by weight of medium per part of 1,2,4-trioxepane starting material.

6. A process for the preparation of lactones according to claim 1 wherein the 1,2,4-trioxepane is added in the pure form if it is a liquid at room temperature, or in the molten state or dissolved in a minimum amount of a suitable solvent if it is a solid at room temperature.

7. A process for the preparation of lactones according to claim 1 wherein the 1,2,4-trioxepane is a reaction product of hexyleneglycol hydroperoxide or isopreneglycol hydroperoxide with a compound selected from the group consisting of cyclobutanone, cyclopentanone, cyclohexanone, cycloheptanone, cyclooctanone, cyclononanone, cyclodecanone, cycloundecanone, cyclododecanone, cyclotridecanone, cyclotetradecanone, cyclopentadecanone, cyclohexadecanone, cycloheptadecanone, cyclooctadecanone, camphor, norbornanone, ethyl2-oxocyclopentylacetate, ethyl6-(2-oxocyclopentyl)hexanoate, 3-methylcyclopentanone, fenchone, 2-methylcyclopentanone, methyl2-cyclopentanonecarboxylate, 4-t-butylcyclohexanone, menthone, 2-methylcyclohexanone, 3-methylcyclohexanone, 2-phenylcyclohexanone, 3,3,5,5-tetramethylcyclohexanone, 2,6-dimethylcyclohexanone, bicylo[3.2.1]octan-2-one, 2B-cyanoethylcyclohexanone, 4-ethylcyclohexanone, bicyclo[3.3.1]nonan-9-one, dihydrocarvone, 2-t-butylcyclohexanone, 3,3,5-trimethylcyclohexanone, 6-carbethoxy-2,6,6-trimethylcyclohexanone, 2,6,6-trimethylcyclohexanone, 2-ethoxycyclohexanone, 2,2,6,6-tetramethylcyclohexanone, 3-methylene-2-norbornanone, pulegone, and ethyl2-oxo-1-cyclooctanecarboxylate.

8. A process for the preparation of lactones according to claim 1 wherein the reaction temperature is maintained between 100 and 300° C.
